(19) United States
(12) Patent Application Publication
(10) Pub. No.: US 2002/0006380 A1
(43) Pub. Date: Jan. 17, 2002

(54) Pyrrole derivatives, their preparation and pharmaceutical compositions containing them

(76) Inventors: Serge Mignani, Chatenay-Malabry (FR); Conception Nemecek, Thiais (FR)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/732,636

(22) Filed: Dec. 8, 2000

Related U.S. Application Data
(63) Continuation of application No. PCT/FR99/01330, filed on Jun. 7, 1999.

(30) Foreign Application Priority Data
Jun. 10, 1998 (FR) 9807274

(51) Int. Cl. A61K 49/00; C07D 471/02; C07D 491/02; C07D 498/02; C07D 513/02; C07D 515/02

(52) U.S. Cl. 424/9.1; 546/121

(57) ABSTRACT

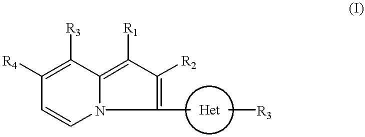

The invention concerns pyrrole derivatives of general formula (I) wherein: R₁ is CONH₂, CN, carboxy, alkyloxycarbonyl, or acyl; R₂ is a H atom, a halogen atom, a CN, alkyl, alkyoxy, alkenyl or trihalogenomethyl radical; R₃ is a H atom or a halogen atom, or an alkyl or OH radical; Het is pyridyl, pyridyl N-oxide or thiazolyl; R₄ is a H atom or a halogen atom, an alkylthio or alkyloxy radical; and R₅ is a H atom, or a hydroxy or alkyloxy radical; provided that when R₃, R₄ and R₅ are H atoms and Het is a pyridin-2-yl radical, then R₁ cannot be acetyl or methyloxycarbonyl and R₂ is a H atom, or R₁ cannot be propionyl and R₂ methyl, the C₁-C₄ alkyl and C₂-C₄ alkenyl radicals being linear or branched, as the case may be in the form of stereoisomers or their mixtures and/or their salts when they exist. Said derivatives are particularly useful for treating and preventing diseases wherein are involved herpes family viruses and/or wherein are involved cytokines including TNFα.

The present invention relates to new pyrrole derivatives of general formula:

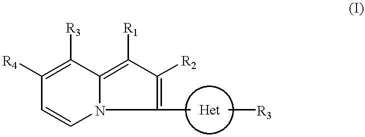

which are useful in the treatment and prevention of conditions in which viruses of the herpes family are involved, and/or in which cytokines, including TNF_{α} (Tumour Necrosis Factor alpha), are involved.

Viruses of the herpes family are responsible for numerous conditions, some of which can be very serious. It comprises in particular the group α, β and γ herpesviruses including the herpes simplex viruses 1 and 2, varicella-zoster, cytomegalovirus (CMV), herpesviruses types 6 and 7 (HHV-6 and HHV-7), Epstein-Barr virus and herpesvirus type 8 (HHV-8). The clinical forms due to a herpes simplex infection can vary from benign forms such as herpes labialis to more serious forms such as genital herpes. Herpes simplex may even be responsible for encephalitis putting the patient's life at risk. Varicella-zoster is the virus responsible for varicella and zona, it may also be responsible for more serious conditions including encephalitis. Cytomegalovirus infections are in general asymptomatic in healthy subjects, but can be the cause of morbidity [retinitis (which may lead to blindness), pneumopathies and the like] and of mortality in immunosuppressed subjects (patients suffering from AIDS or any other immunodeficiency, for example after organ transplantation or after anticancer chemotherapy). The cytomegalovirus is also responsible for severe clinical manifestations for the foetus or newborn in the case of a primary infection during pregnancy or during seropositive blood transfusion into a seronegative newborn. The herpesviruses HHV-6 and -7 are responsible for roseola and can be reactivated in immunosuppressed subjects. The HHV-8 virus is involved in Kaposi's sarcoma.

The treatments existing up until now are likely to cause serious side effects. Furthermore, for some of these viruses, the treatments can most often only be used by the intravenous route.

Cytokines (including TNF_{α}) are capable of activating various viruses and/or retroviruses, for example the cytomegalovirus or the Human Immunodeficiency Virus (HIV), and are also capable of activating cellular genes, in particular those involved in inflammatory processes, such as the genes for chemokines, cytokines and adhesion molecules.

In European Applications EP 118 321, EP 147 317 and EP 124 384 and in French Application 2 539 417, there have been described pyrrole derivatives having an antithrombotic activity or serving as intermediates for the preparation of antithrombotic derivatives. Pyrrole derivatives having an inhibitory activity on the effects of TNF_{α} have been described in French Application 2 735 476.

In the general formula (I):

R₁ is a carboxamide, cyano, carboxyl, alkyloxycarbonyl or acyl radical,

R₂ is a hydrogen or halogen atom, or a cyano, alkyl, alkyloxy, alkenyl or trihalomethyl radical,

R₃ is a hydrogen or halogen atom, or an alkyl or hydroxyl radical,

Het is a pyridyl, pyridyl N-oxide or thiazolyl radical,

R₄ is a hydrogen or halogen atom, or an alkylthio or alkyloxy radical, and

R₅ is a hydrogen atom, or a hydroxyl or alkyloxy radical,

it being understood that when R₃, R₄ and R₅ are hydrogen atoms and Het is a 2-pyridyl radical, then R₁ cannot be acetyl or methyloxycarbonyl and R₂ a hydrogen atom, or alternatively R₁ cannot be propionyl and R₂ methyl, the alkyl radicals being straight or branched and containing 1 to 4 carbon atoms and the alkenyl radicals being straight or branched and containing 2 to 4 carbon atoms.

According to the invention, the halogen atoms are chosen from fluorine, chlorine, bromide or iodine.

According to the invention, the preparation of the products of general formula (I) is carried out by preparing a nitrile intermediate of general formula:

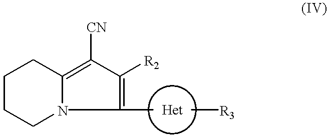

in which Het and R₃ are defined as above, and R₂ is a hydrogen atom or an alkyl or alkyloxy radical, by the action of an acrylic derivative of general formula:

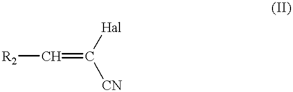

in which R₂ is defined as above, and Hal is a halogen atom (for example a chlorine atom) on an acid of general formula:

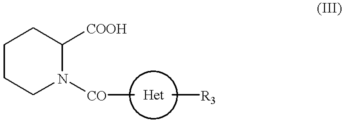

in which Het and R₃ are defined as above, followed by the steps of introducing, where appropriate, the radical R₂, aromatization, and introducing the radicals R₄ and/or R₅, and/or where appropriate converting the nitrile to an amide, an acid, an ester or an acyl radical, or alternatively, where appropriate, converting the ester radical to an acid or to an acyl radical, by any known methods which do not alter the rest of the molecule.

By way of example, the known methods may be in particular methods described in the patent applications cited above, or the methods described in the examples which follow, or methods analogous to these methods.

The reaction of the product of general formula (II) with the acid of general formula (III) is generally carried out using the acid salt (sodium salt for example), in acetic anhydride at a temperature of between 80 and 130° C.

When it is desired to obtain the derivative for which R₂ represents cyano, the product obtained is subjected to treatment with isocyanatosulphonyl chloride at a temperature of between 0 and 85° C. in an organic solvent such as acetonitrile.

When it is desired to obtain the derivative for which R₂ represents trihalomethyl or alkenyl, the derivative of general formula (IV) obtained is treated beforehand according to the method described in French Application 2 735 476, or by any method analogous thereto.

When it is desired to obtain a derivative for which R₂ is a halogen atom, the product of general formula (IV) is subjected in the first place to a treatment with N-halosuccinimide, followed by a reduction of the product obtained, for example by catalytic hydrogenation in an acidic medium, in the presence of palladium on carbon. The procedure is advantageously carried out with 1, 3 or 5 equivalents of N-halosuccinimide (depending on the substituents R₄ and R₅ which are desired subsequently), in a chlorinated solvent (dichloromethane or dichloroethane for example) or in a nitrile (acetonitrile for example) at the reflux temperature of the reaction mixture.

In particular, when it is desired to obtain a derivative of general formula (I) for which R₄ and R₅ are simultaneously hydrogen, the substituent R₂ is introduced where appropriate starting with the intermediate of general formula (IV) according to the methods described above or by any other similar method, and then the aromatization is carried out by a treatment with selenium oxide SeO₂, with DDQ or with chloranil for example. The procedure is advantageously carried out by the action of selenium oxide in a solvent, for example dioxane or xylene, at the reflux temperature of the mixture.

In particular, when it is desired to obtain a derivative of general formula (I) in which R₄ is a hydrogen atom, and R₅ is defined as above with the exception of representing a hydrogen atom, and R₂ is halogen, the halogenation of the intermediate of general formula (IV) is carried out with an N-halosuccinimide, followed by the reduction of the product obtained, for example by catalytic hydrogenation in an acidic medium, in the presence of palladium on carbon, so as to obtain a derivative of general formula (V):

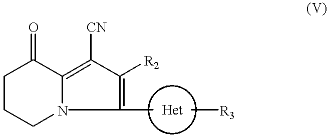

in which Het and R₃ are defined as above, and R₂ is a halogen atom. The bromination of the derivative of general formula (V) is then carried out, followed by a dehydrohalogenation so as to obtain a derivative for which R₅ is a hydroxyl radical, optionally followed by the conversion of the hydroxyl derivative to an alkyloxy.

The halogenation is carried out with five equivalents of N-halosuccinimide. The procedure is advantageously carried out in a chlorinated solvent (dichloromethane or dichloroethane for example) or in a nitrile (acetonitrile for example) at the reflux temperature of the reaction mixture.

The bromination is advantageously carried out by addition of bromine at a temperature of between 15° C. and 30° C.

The dehydrohalogenation may be carried out with an alkali metal bromide, for example lithium bromide, in dimethylformamide at the reflux temperature of the mixture.

The conversion of the hydroxyl group to an alkyloxy is advantageously carried out by the action of a haloalkyl compound in the presence of an alkali metal hydride, for example sodium hydride. Preferably, the halogen atom is chosen from iodine, bromine or chlorine.

In particular, when it is desired to obtain a derivative of general formula (I) for which R₄ is different from hydrogen, R₅ is a hydrogen atom, and R₂ is a halogen atom, a halogenation of the intermediate of general formula (IV) is carried out with an N-halosuccinimide, followed by the reduction of the product obtained, for example by catalytic hydrogenation in acidic medium, in the presence of palladium, so as to obtain a derivative of general formula (VIII):

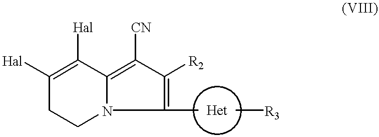

for which Het and R₃ are defined as above, and R₂ and Hal are halogen atoms, and then a treatment is carried out, where appropriate, with an alcoholate or a thiolate, and finally the aromatization.

The halogenation is carried out with equivalents of N-halosuccinimide. The procedure is advantageously carried out in a chlorinated solvent (dichloromethane or dichloroethane for example) or in a nitrile (acetonitrile for example) at the reflux temperature of the reaction mixture.

The treatment with an alcoholate or with a thiolate (for example sodium methoxide or sodium thiomethoxide) is carried out in toluene at the reflux temperature of the reaction mixture.

The aromatization is advantageously obtained by addition of a base to the reaction mixture, for example potassium hydroxide.

In particular, when it is desired to obtain a derivative of general formula (I) for which R₄ is different from hydrogen, R₅ is a hydrogen atom, R₂ is different from a halogen atom, the substituent R₂ is introduced beforehand according to the methods described above or by any similar method, and then the halogenation of the derivative obtained is carried out with an N-halosuccinimide, so as to obtain an intermediate of general formula (VI):

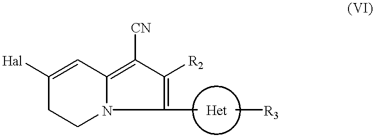

for which Het, R₃ and Hal are defined as above, and R₂ is different from halogen, and then where appropriate a treatment is carried out with a thiolate or an alcoholate, and finally the aromatization is carried out.

The halogenation is carried out with 2 equivalents of N-halosuccinimide. The procedure is advantageously carried out in a chlorinated solvent (dichloromethane or dichloroethane for example) or in a nitrile (acetonitrile for example) at the reflux temperature of the reaction mixture.

The treatment with an alcoholate or with a thiolate (for example sodium methoxide or sodium thiomethoxide) is carried out in toluene at the reflux temperature of the reaction mixture.

The aromatization is advantageously obtained by addition of a base to the reaction mixture, for example potassium hydroxide.

In particular, when it is desired to obtain a derivative of general formula (I) for which R₄ and R₅ are different from hydrogen, a halogenation of the intermediate of general formula (IV) is carried out with an N-halosuccinimide so as to obtain the derivative of general formula:

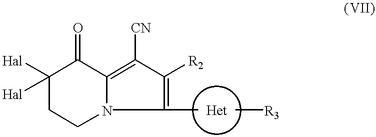

for which Het and R₃ are defined as above, and R₂ and Hal are halogen atoms, followed by a treatment in a basic medium.

The halogenation is carried out with 5 equivalents of N-halosuccinimide. The procedure is advantageously carried out in a chlorinated solvent (dichloromethane or dichloroethane for example) or in a nitrile (acetonitrile for example) at the reflux temperature of the reaction mixture.

In the case when it is desired that the substituent R₂ is not a halogen atom, the introduction of the substituent R₂ is carried out beforehand according to the methods described above, followed by the halogenation of the intermediate obtained with an N-halosuccinimide. The halogenation is carried out in this case with 4 equivalents of N-halosuccinimide, preferably in a chlorinated solvent (dichloromethane or dichloroethane for example) or in a nitrile (acetonitrile for example) at the reflux temperature of the reaction mixture.

The treatment in a basic medium is similar to the methods described in the literature: Synth. Comm., 1995, 25, 2337, or alternatively JACS, 1957, 79, 1205. In particular, the procedure is carried out in the presence of an alcoholate (for example potassium tert-butoxide or sodium methoxide) at the reflux temperature of the mixture.

The hydrolysis of the nitrile at the 1-position to an amide is carried out according to known methods, in particular by heating in an alkaline medium in an organic solvent such as for example tert-butanol at a temperature of between 30° C. and 85° C., or in a concentrated acidic medium at a temperature of between 20° C. and 100° C.

The hydrolysis of the ester or of the nitrile to an acid is carried out according to known methods, in particular in a basic medium in an alcohol with a high boiling point, for example in the presence of potassium hydroxide in ethylene glycol, at a temperature of between 100° C. and the reflux temperature of the reaction mixture.

The conversion of the acid functional group to an alkyloxycarbonyl radical is carried out by the usual esterification methods which do not alter the rest of the molecule, in particular by application or by adaptation of the methods described in Tetrahedron, 33, 683 (1977), Tetrahedron Letters, 4475 (1978) or Bull. Soc. Chim. Japan, 40, 2380 (1967).

The conversion to the acyl radical is carried out using, as starting material, the derivative for which R₁ is carboxyl, by preparing the acid halide and then by the action of a malonic derivative (for example methyl malonate), followed by decarboxylation of the derivative obtained. The procedure is carried out under the conditions described or by analogy with the conditions described in Tetrahedron, 14, 321 (1961); Org. Synth., 3, 169; J. Org. Chem., 50, 2622 (1987); Synthesis, 284 (1982).

The oxidation of the pyridyl radical to pyridyl N-oxide is carried out by any oxidation method which does not alter the rest of the molecule. In particular, the procedure is carried out by means of a peracid such as m-chloroperbenzoic acid, in an alcoholic medium (ethanol for example) at a temperature of between 15 and 30° C.

When the derivative of formula (IV) is treated with 5 equivalents of N-halosuccinimide and then a catalytic hydrogenation is carried out, a mixture of the products of formulae (V) and (VIII) is obtained, which products are then separated by known methods, in particular by chromatography.

It is understood that the present invention also relates to the stereoisomers of the products of general formula (I) when these exist, as well as mixtures thereof.

The products according to the invention which carry an amino or alkylamino radical may be converted to acid addition salts by known methods. It is understood that these salts are also within the scope of the present invention.

As examples of addition salts with pharmaceutically acceptable acids, there may be mentioned the salts formed with inorganic acids (hydrochlorides, hydrobromides, sulphates, nitrates, phosphates) or with organic acids (succinates, fumarates, tartrates, acetates, propionates, maleates, citrates, methanesulphonates, p-toluenesulphonates, isethionates and the like), or with substitution derivatives of these compounds.

The present invention also relates to the medicaments formed by the pyrrole derivatives of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl.

The action of the derivatives of general formula (I) on viruses of the herpes family has been demonstrated in the techniques described by NEYTS et al., Virology, 179, 41-50 (1990); Andrei et al., Eur. J. Clin. Microbiol. Infect. Dis., 10, 1026-1033 (1991); or in the technique described by Andrei et al., Eur. J. Clin. Microbiol. Infect. Dis., 11, 143-151 (1992), Reymen et al., Antiviral Res., 28, 343-357 (1995).

The technique used consists in the measurement of the cytopathogenic effect of the virus and of its protection by the use of the products of general formula (I). The antiviral activity is assessed by the measurement of the IC₅₀ (concentration necessary to inhibit 50% of the cytopathogenic effect induced by the virus).

The activity of the products according to the invention on the cytomegalovirus has been studied on the Davis and AD-169 strains. On the Davis strain, the products according to the invention proved active at IC₅₀ values of between 0.005 μg/ml and 15 μg/ml, and on the AD-169 strain, the products according to the invention proved active at IC₅₀ values of between 0.01 and 15 μg/ml.

Moreover, no product manifests a cytotoxic effect at the dose of 15 μg/ml.

The inhibitory activity of the derivatives according to the invention towards TNF_{α} has been demonstrated in the following manner: the effects of the derivatives according to the invention on the reactivation of the HIV virus by TNF_{α} (10 Units/ml) or Phorbol Myristate Acetate (PMA at 10-7 M) were studied in U1 cells derived from the promonocytic line U937 [Folks et al., Science, 238, 800 (1987)].

Experimental Study of the Inhibitory Activity Towards TNF_{α}

The product to be studied is dissolved in dimethylformamide (DMF) or dimethyl sulphoxide (DMSO). The stock solutions are stored at a temperature of 4° C. and diluted in culture medium on the day of the experiment so that the solvent concentration is constant (0.1%).

The U1 cells are pretreated 5 hours before stimulation with product concentrations ranging from 0.001 μM to 10 μM. Three days after induction, the viral supernatant is collected and the reverse transcriptase activity reflecting viral production is evaluated (SPA test).

The reverse transcriptase activity is measured by known techniques, in duplicate [Strebel et al., Nature, 328, 728 (1987)].

Some controls do not receive the activator. Other controls do not receive the product to be studied. Others receive neither the product nor the activator.

Results:

The decrease in viral production caused by the derivatives according to the invention is significant and dose-dependent in the case of U1 cells treated with TNF_{α} or with PMA. On day 3, a decrease of at least 50% in the production of reverse transcriptase is observed for the U1 cells treated with 10 units/ml of TNF_{α} and supplemented with a concentration of 10 μM of the products.

Moreover, no cytotoxicity of the test products is observed on the viability of the cells at the concentration of 1 μM.

In this method, the compounds according to the invention have proved active at concentrations of between 0.01 μM and 10 μM.

The pharmaceutical compositions containing the pyrrole derivatives of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, are particularly advantageous because of the fact that they find applications in many diseases of viral origin, particularly retinitis, pneumopathies, encephalitis, digestive infections and encephalitis caused by CMV, Kaposi's sarcoma, herpes labialis, genital herpes, herpetic encephalitis, varicella, roseola, zonas, hepatitis (caused by cytomegalovirus), ophthalmic infections or in the prophylaxis of the infection or of the viral reactivation. They may also be highly advantageous in the treatment and prevention of cardiovascular diseases, particularly in restenosis which may follow an angioplasty.

Likewise, the pharmaceutical compositions containing pyrrole derivatives of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, are also particularly advantageous because of the fact that they find applications in any of the pathologies involving cytokines including TNF_{α}. By way of example, there may be mentioned: osteoarticular diseases of inflammatory origin, asthma, diabetes, cachexia (secondary to an infection or to a tumour), diseases of the digestive system such as Crohn's disease and ulcerohaemorrhagic rectocolitis, disorders of the central and/or peripheral nervous system, immunological diseases including graft-versus-host disease and allograft rejection, lesions due to perfusion and/or ischaemia, and viral or infectious diseases including pathologies related to HIV and to tuberculosis.

They are also advantageous for their applications in pathologies related to IL-8 reactivated by TNF_{α}, such as psoriasis, inflammatory diseases of the digestive tube, respiratory distress syndrome, asthma, lesions induced by a perfusion, thrombosis, glomerulonephritis, and inflammatory osteoarticular pathologies.

They can also be used in pathologies involving adhesion molecules, for example diseases of the cardiovascular system (in particular artherosclerosis or thrombosis), lesions related to ischaemia-reperfusion, neurological disorders, digestive, pulmonary or articular inflammatory pathologies, immunological diseases including graft rejection.

The following examples, given with no limitation being implied, illustrate the invention.

### EXAMPLE 1

2-Chloro-3-pyridin-3-ylindolizine-1-carbonitrile is prepared according to the following method:

1.5 g of selenium oxide are added to 0.5 g of 2-chloro-3-pyridin-3-yl-5,6,7,8-tetrahydroindolizine-1-carbonitrile in solution in 50 cm³ of xylene. The reaction mixture is heated at the reflux temperature of xylene for 24 hours and then filtered on Celite and evaporated to dryness under reduced pressure (2.7 kPa) at a temperature close to 60° C. The product obtained is taken up in 50 cm³ of water and extracted with 3 times 50 cm³ of dichloromethane. The organic phases are combined and washed with twice 50 cm³ of water and then dried over magnesium sulphate and concentrated to dryness under reduced pressure (2.7 kPa) at a temperature close to 60° C. 0.78 g of an orange-coloured solid is obtained, which solid is recrystallized from 20 cm³ of isopropanol. 0.165 g of 2-chloro-3-pyridin-3-ylindolizine-1-carbonitrile is obtained in the form of a beige solid melting at 184° C.

2-Chloro-3-pyridin-3-yl-5,6,7,8-tetrahydroindolizine-1-carbonitrile is prepared according to the following method:

2.23 cm³ of hydrochloric acid (1 N) are added to a suspension of 7.41 g of 2,7-dichloro-3-pyridin-3-yl-5,6-dihydroindolizine-1-carbonitrile in 750 cm³ of ethanol and 120 cm³ of acetic acid. A clear yellow solution is obtained. After having purged with argon, 2.62 g of 10% palladium on carbon are added and then a hydrogen stream is passed through for 2 hours. The reaction mixture is filtered on Celite and the cake is washed with 200 cm³ of an ethanol/acetic acid (2/1) mixture. The filtrate is evaporated to dryness under reduced pressure (2.7 kPa) at a temperature close to 60° C. The yellow oil obtained is taken up in 200 cm³ of water and the pH is brought to 8-9 by addition of powdered sodium hydrogen carbonate. The mixture is filtered and the precipitate is washed with twice 20 cm³ of water. 5.6 g of a white solid are obtained, which solid is chromatographed on a column 5.7 cm in diameter containing 800 g of silica (0.02-0.045). The elution is carried out with dichloromethane, at a pressure of 150 kPa, collecting 50 cm³ fractions. The homogeneous fractions are pooled and concentrated to dryness under reduced pressure (2.7 kPa) at a temperature close to 40° C. 4.13 g of 2-chloro-3-pyridin-3-yl-5,6,7,8-tetrahydroindolizine-1-carbonitrile are thus obtained in the form of a yellowish white solid melting at 151° C.

2,7-Dichloro-3-pyridin-3-yl-5,6-dihydroindolizine-1-carbonitrile is prepared according to the following method:

18.55 g of N-chlorosuccinimide are added to 10 g of 3-pyridin-3-yl-5,6,7,8-tetrahydroindolizine-1-carbonitrile in solution in 1000 cm³ of acetonitrile. The mixture is heated at the reflux temperature of acetonitrile for 1 hour. The reaction mixture is evaporated to dryness under reduced pressure (2.7 kPa) at a temperature close to 60° C. The yellow oil obtained is taken up in 200 cm³ of water and the pH is brought to 8-9 by addition of powdered sodium hydrogen carbonate. The mixture is filtered and washed with twice 30 cm³ of water. 13 g of a yellow solid are obtained, which solid is chromatographed on a column 8 cm in diameter containing 1500 g of silica (0.02-0.045). The elution is carried out with a dichloromethane/methanol (98/2) mixture, at a pressure of 150 kPa, collecting 50 cm³ fractions. The homogeneous fractions are pooled and evaporated to dryness under reduced pressure (2.7 kPa) at a temperature close to 40° C. 7.41 g of 2,7-dichloro-3-pyridin-3-ylindolizine-1-carbonitrile are thus obtained in the form of a pale yellow solid melting at 216° C.

3-Pyridin-3-yl-5,6,7,8-tetrahydroindolizine-1-carbonitrile is described in patent application EP 124 384.

### EXAMPLE 2

2-Chloro-3-pyridin-3-ylindolizine-1-carboxamide is prepared according to the following method:

A mixture of 1.69 g of 2-chloro-3-pyridin-3-ylindolizine-1-carbonitrile and 1.3 g of potassium hydroxide in 100 cm³ of tert-butanol is heated at the reflux temperature of tert-butanol for 18 hours. The reaction mixture is evaporated to dryness under reduced pressure (2.7 kPa) at a temperature close to 60° C. The brown solid obtained is taken up in 100 cm³ of water and then filtered and washed with twice 10 cm³ of water. 1.6 g of an orange-coloured solid are obtained, which solid is recrystallized from 130 cm³ of isopropanol. 0.869 g of 2-chloro-3-pyridin-3-ylindolizine-1-carboxamide is thus obtained in the form of a white solid melting at 234° C.

2-Chloro-3-pyridin-3-ylindolizine-1-carbonitrile is prepared as described in Example 1.

### EXAMPLE 3

3-Pyridin-3-ylindolizine-1-carboxamide is prepared according to the following method:

A mixture of 0.95 g of 3-pyridin-3-ylindolizine-1-carbonitrile and 0.86 g of potassium hydroxide in 20 cm³ of tert-butanol is heated at the reflux temperature of tert-butanol for 5 hours. The reaction mixture is evaporated to dryness under reduced pressure (2.7 kPa) at a temperature close to 60° C. The orange-coloured solid obtained is taken up in 100 cm³ of water and then filtered and washed with twice 10 cm³ of water. 0.9 g of an orange-coloured solid is obtained, which solid is recrystallized from 23 cm³ of acetonitrile. 0.36 g of 3-pyridin-3-ylindolizine-1-carboxamide is thus obtained in the form of a pale yellow solid melting at 160° C.

3-Pyridin-3-ylindolizine-1-carbonitrile is prepared according to the following method:

A suspension of 1.42 g of 3-pyridin-3-yl-5,6,7,8-tetrahydroindolizine-1-carbonitrile in solution in 50 cm³ of xylene is heated at 80° C. and then 4.93 g of selenium oxide are added. A clear solution is obtained. The reaction mixture is heated at the reflux temperature of xylene for 4 hours and then filtered on Celite, and evaporated to dryness under reduced pressure (2.7 kPa), at a temperature close to 60° C. The brown-orange-coloured solid obtained is taken up in 50 cm³ and then washed with twice 10 cm³ of water. 0.95 g of 3-pyridin-3-ylindolizine-1-carbonitrile is thus obtained in the form of an orange-brown solid (Rf=0.73; eluent: 90/10 dichloromethane/methanol).

3-Pyridin-3-yl-5,6,7,8-tetrahydroindolizine-1-carbonitrile is described in patent application EP 124 384.

### EXAMPLE 4

2,7-Dichloro-3-pyridin-3-ylindolizine-1-carboxamide is prepared according to the following method:

A brown solution of 0.477 g of 2,7-dichloro-3-pyridin-3-ylindolizine-1-carbonitrile in 29 cm³ of 60% sulphuric acid is heated at the reflux temperature of the solvent for 2 hours and 30 minutes. The reaction mixture is added to 60 g of ice, treated with 3S black, filtered on sintered glass, clogged with 3S black and then washed with twice 3 cm³ of water. The pH of the filtrate is brought to 8-9 by addition of aqueous ammonia (28%). A yellow suspension is thus obtained to which 60 cm³ of water are added. The mixture is filtered and washed with twice 5 cm³ of water. A cream-coloured powder is obtained which is recrystallized from 16 cm³ of ethanol. 0.19 g of 2,7-dichloro-3-pyridin-3-ylindolizine-1-carboxamide is thus obtained in the form of a cream-coloured powder melting at 228° C.

2,7-Dichloro-3-pyridin-3-ylindolizine-1-carbonitrile is prepared according to the following method:

1.5 g of 2,7,8-trichloro-3-pyridin-3-yl-5,6-dihydroindolizine-1-carbonitrile dihydrochloride salt in solution in 30 cm³ of a mixture of potassium hydroxide in ethanol (0.8 M) are heated at the reflux temperature of the solvent for 3 hours. A brown suspension is obtained to which 75 cm³ of water are added. The mixture is filtered and the cake is washed with twice 15 cm³ of water. A beige powder is obtained which is recrystallized from 40 cm³ of ethanol. 0.185 g of 2,7-dichloro-3-pyridin-3-ylindolizine-1-carbonitrile is thus obtained in the form of a beige powder melting at 248° C.

2,7,8-Trichloro-3-pyridin-3-yl-5,6-dihydroindolizine-1-carbonitrile dihydrochloride salt is prepared according to the following method:

5.3 cm³ of hydrochloric acid (10 N) are added to a suspension of 21.4 g of a mixture containing 2,7,7-trichloro-8-oxo-3-pyridin-3-yl-5,6-dihydroindolizine-1-carbonitrile at about 80 mol % and 2,7,8-trichloro-3-pyridin-3-yl-5,6-dihydroindolizine-1-carbonitrile at about 20 mol % in 2100 cm³ of ethanol and 640 cm³ of acetic acid. A brown solution is obtained. 6.3 g of 10% palladium on carbon are added. After having purged with argon, a hydrogen stream is passed through for 1 hour and 20 minutes. The reaction mixture is filtered on Celite and the cake is washed with twice 100 cm³ of an ethanol/acetic acid (2/1) mixture. The brown filtrate obtained is evaporated to dryness under reduced pressure (2.7 kPa) at a temperature close to 60° C. A brown wax is obtained which is chromatographed on a column 4 cm in diameter containing 250 g of silica (0.02-0.045). The elution is carried out with a mixture of dichloromethane/methanol (90/10), collecting 20 cm³ fractions. The homogeneous fractions are pooled and concentrated to dryness under reduced pressure (2.7 kPa) at a temperature close to 50° C. 3.5 g of 2,7,8-trichloro-3-pyridin-3-yl-5,6-dihydroindolizine-1-carbonitrile dihydrochloride salt are thus obtained in the form of a beige powder melting at 240° C.

The mixture containing 2,7,7-trichloro-8-oxo-3-pyridin-3-yl-5,6-dihydroindolizine-1-carbonitrile at about 80 mol % and 2,7,8-trichloro-3-pyridin-3-yl-5,6-dihydroindolizine-1-carbonitrile at about 20 mol % is prepared according to the following method:

56.9 g of N-chlorosuccinimide are added to 19 g of 3-pyridin-3-yl-5,6,7,8-tetrahydroindolizine-1-carbonitrile in solution in 1540 cm³ of acetonitrile. The mixture is heated at the reflux temperature of acetonitrile for 4 hours and 30 minutes. The reaction mixture is evaporated to dryness under reduced pressure (2.7 kPa) at a temperature close to 50° C. The orange-coloured solid obtained is taken up in 150 cm³ of water and the pH is brought to 8-9 by addition of powdered sodium hydrogen carbonate (33 g). The mixture is filtered and washed with twice 25 cm³ of water. 26.66 g of a brick red powder are obtained. This powder is taken up in 100 cm³ of water, filtered and washed with twice 25 cm³ of water. 21.4 g of a mixture containing 2,7,7-trichloro-8-oxo-3-pyridin-3-yl-5,6-dihydroindolizine-1-carbonitrile at about 80 mol % and 2,7,8-trichloro-3-pyridin-3-yl-5,6-dihydroindolizine-1-carbonitrile at about 20 mol % are thus obtained in the form of a brown powder melting at 180° C. 3-Pyridin-3-yl-5,6,7,8-tetrahydroindolizine-1-carbonitrile is described in patent application EP 124,384.

### EXAMPLE 5

Methyl 2-methyl-3-pyridin-3-ylindolizine-1-carboxylate methane sulphonate salt is prepared according to the following method:

0.16 g of methyl 2-methyl-3-pyridin-3-yl-indolizine-1-carboxylate in solution in 1 cm³ of ethanol is cooled to 0° C. and 0.3 cm³ of a methanesulphonic acid solution in ethanol (4.1 N) is added, followed by 1 cm³ of ethanol. The temperature of the reaction mixture is kept at 0° C. for 3 hours. The mixture is filtered and 0.0395 g of methyl 2-methyl-3-pyridin-3-ylindolizine-1-carboxylate methane sulphonate salt is thus obtained in the form of a yellow solid melting at 111° C.

Methyl 2-methyl-3-pyridin-3-ylindolizine-1-carboxylate is prepared according to the following method:

2.87 g of selenium oxide are added to 1 g of methyl 2-methyl-3-pyridin-3-yl-5,6,7,8-tetrahydroindolizine-1-carboxylate in solution in 100 cm³ of xylene. The reaction mixture is heated at 100° C. for 27 hours and then it is filtered on Celite, and evaporated to dryness under reduced pressure (2.7 kPa) at a temperature close to 60° C. The product obtained is taken up in 100 cm³ of water and the pH is brought to 8-9 by addition of sodium hydrogen carbonate, and then the mixture is extracted with 3 times 100 cm³ of dichloromethane. The organic phases are pooled and washed with twice 100 cm³ of water and then dried over magnesium sulphate and evaporated to dryness under reduced pressure (2.7 kPa) at a temperature close to 60° C. 0.34 g of an orange-coloured lacquer is obtained which is chromatographed on a column 4 cm in diameter containing 90 g of silica (0.02-0.045). The elution is carried out with dichloromethane, collecting 100 cm³ fractions. The homogeneous fractions are pooled and evaporated to dryness under reduced pressure (2.7 kPa) at a temperature close to 40° C. 0.1 g of a yellow oil is obtained which is taken up in 5 cm³ of diisopropyl ether. 0.1 g of methyl 2-methyl-3-pyridin-3-yl-indolizine-1-carboxylate is thus obtained in the form of a white solid (Rf=0.47, eluent: 80/20 dichloromethane/ethyl acetate).

Methyl 2-methyl-3-pyridin-3-yl-5,6,7,8-tetra-hydroindolizine-1-carboxylate is prepared according to the following method:

6.9 cm³ of triethylamine are added to a cream-coloured suspension of 11 g of sodium N-nicotinoylpiperidine-2-carboxylate in 80 cm³ of 1,2-dichloroethane; a cream-coloured suspension is obtained which is kept stirred at room temperature for 1 hour. 20.4 cm³ of methyl 2-methyl-2-bromobutenoate in solution in 90 cm³ of 1,2-dichloroethane are added to a clear solution of 9 g of para-toluenesulphonyl chloride in 80 cm³ of 1,2-dichloroethane. A clear yellow solution is obtained. This solution is added dropwise to the cream-coloured suspension obtained above. The reaction mixture is cooled to 0° C. and then 36 cm³ of triethylamine are added and the mixture is allowed to return to room temperature. The reaction mixture is kept stirred and at room temperature for 12 hours and then it is heated at 55° C. for 7 hours. The reaction mixture is washed with 3 times 250 cm³ of water and dried over magnesium sulphate. It is evaporated to dryness under reduced pressure (2.7 kPa) at a temperature close to 60° C. 31.06 g of a black oil are obtained, which oil is chromatographed on a column 7.5 cm in diameter containing 1950 g of silica (0.02-0.045). The elution is carried out with a mixture of dichloromethane/ethyl acetate (80/20), at a pressure of 150 kPa, collecting 70 cm³ fractions. The homogeneous fractions are pooled and evaporated to dryness under reduced pressure (2.7 kPa) at a temperature close to 40° C. 1.08 g of methyl 2-methyl-3-pyridin-3-yl-5,6,7,8-tetrahydroindolizine-1-carboxylate are thus obtained in the form of a brown solid melting at 68° C. (gum).

The sodium salt of N-nicotinoylpiperidine-2-carboxylic acid is prepared according to the following method:

18.6 cm³ of sodium hydroxide (10 N) are added to a solution of 44.48 g of ethyl N-nicotinoylpiperidine-2-carboxylate in 420 cm³ of acetonitrile and 42 cm³ of methanol. The reaction mixture is filtered and the cake is washed with twice 50 cm³ of diethyl ether. 35 g of the sodium salt of N-nicotinoylpiperidine-2-carboxylic acid are thus obtained in the form of a white solid melting at a temperature of greater than 260° C.

Ethyl N-nicotinoylpiperidine-2-carboxylate is prepared as described in patent application EP 124,384.

### EXAMPLE 6

2-Chloro-8-hydroxy-3-pyridin-3-ylindolizine-1-carboxamide is prepared according to the following method:

A mixture of 2 g of 7-bromo-2-chloro-3-pyridin-3-yl-8-oxo-5,6-dihydroindolizine-1-carboxamide and 2.36 g of lithium bromide in 20 cm³ of dimethylformamide is heated at 130° C. for 40 minutes. The reaction mixture is evaporated to dryness under reduced pressure (2.7 kPa) at a temperature close to 60° C. The orange-coloured oil obtained is taken up in 100 cm³ of water and the pH is brought to 9 by addition of sodium hydrogen carbonate. The mixture is extracted with 100 cm³ of dichloromethane. An emulsion is obtained which is filtered. The brown cake is taken up in 6 times 100 cm³ of a mixture of dichloromethane/methanol (50/50). The organic phases are dried over magnesium sulphate and evaporated to dryness under reduced pressure (2.7 kPa) at a temperature close to 50° C. The product obtained is dissolved in 200 cm³ of a dichloromethane/methanol (50/50) mixture and then bound to 7 g of silica (0.02-0.04), and chromatographed on a column 5 cm in diameter containing 600 g of silica (0.02-0.045). The elution is carried out with a mixture of dichloromethane/methanol (95/5), collecting 80 cm³ fractions. The homogeneous fractions are pooled and evaporated to dryness under reduced pressure (2.7 kPa) at a temperature close to 40° C. 0.51 g of a cream-coloured crystallized solid is obtained which is recrystallized from 120 cm³ of 1-butanol. 0.377 g of 2-chloro-8-hydroxy-3-pyridin-3-ylindolizine-1-carboxamide is thus obtained in the form of a white powder melting at a temperature of greater than 260° C.

7-Bromo-2-chloro-3-pyridin-3-yl-8-oxo-5,6-dihydroindolizine-1-carboxamide is prepared according to the following method:

1.77 cm³ of bromine in solution in 160 cm³ of acetic acid are added, over 2 hours, to a mixture of 10 g of 2-chloro-3-pyridin-3-yl-8-oxo-5,6,7-trihydroindolizine-1-carboxamide in 800 cm³ of acetic acid. The reaction mixture is kept at room temperature for 82 hours. The solvent is evaporated to dryness under reduced pressure (2.7 kPa) at a temperature close to 55° C. The brown residue obtained is taken up in 250 cm³ of water and the pH is brought to 1 by addition of 25 cm³ of hydrochloric acid (4 N). The mixture is filtered and the pH of the filtrate is brought to 8-9 by addition of sodium hydrogen carbonate. 11.2 g of 7-bromo-2-chloro-3-pyridin-3-yl-8-oxo-5,6-dihydroindolizine-1-carboxamide are thus obtained in the form of an ochre-coloured powder melting at 259° C. (decomposition).

2-Chloro-3-pyridin-3-yl-5,6,7-trihydro-8-oxoindolizine-1-carboxamide is prepared according to the following method:

12.1 g of 2-chloro-3-pyridin-3-yl-5,6,7-trihydro-8-oxo-indolizine-1-carbonitrile in 82 cm³ of 60% sulphuric acid are heated at 97° C. for 2 hours. The reaction mixture is poured over 450 g of an ice/water mixture and the pH is brought to 9-10 by addition of aqueous ammonia (28%). The precipitate is filtered and then taken up in 700 cm³ of dichloromethane. The organic phase is dried over magnesium sulphate and then evaporated to dryness under reduced pressure (2.7 kPa) at a temperature close to 50° C. 10 g of 2-chloro-3-pyridin-3-yl-5,6,7-trihydro-8-oxoindolizine-1-carboxamide are thus obtained in the form of an ochre-coloured crystallized product melting at 245° C.

2-Chloro-3-pyridin-3-yl-5,6,7-trihydro-8-oxoindolizine-1-carbonitrile is prepared according to the following method:

4.99 g of palladium on carbon (10%) in suspension in 100 cm³ of ethanol are added to a mixture of 16 g of 2,7,7-trichloro-8-oxo-3-pyridin-3-yl-5,6,7,8-tetrahydroindolizine-1-carbonitrile, 1500 cm³ of ethanol, 500 cm³ of acetic acid and 4 cm³ of hydrochloric acid (12 N). A hydrogen stream is passed through for 3 hours. The reaction mixture is filtered on Celite and then evaporated to dryness under reduced pressure (2.7 kPa) at a temperature close to 60° C. The light brown solid obtained is taken up in 150 cm³ of water and the pH is brought to 8-9 by addition of sodium hydrogen carbonate, and then filtered and washed with twice 20 cm³ of water. 12.1 g of 2-choro-3-pyridin-3-yl-5,6,7-trihydro-8-oxoindolizine-1-carbonitrile are thus obtained in the form of a dark beige powder melting at 264° C.

2,7,7-Trichloro-8-oxo-3-pyridin-3-yl-5,6,7,8-tetrahydroindolizine-1-carbonitrile is prepared as described in Example 4.

### EXAMPLE 7

2-Chloro-3-(5-bromopyridin-3-yl)indolizine-1-carboxamide is prepared as described in Example 2 but starting with 0.8 g of 2-chloro-3-(5-bromopyridin-3-yl)indolizine-1-carbonitrile and 1.6 g of potassium hydroxide in 60 cm³ of tert-butanol. 0.07 g of 2-chloro-3-(5-bromopyridin-3-yl)indolizine-1-carboxamide is thus obtained in the form of a cream-coloured solid melting at more than 260° C. (Rf=0.53; thin-layer chromatography on silica gel; eluent dichloromethane/methanol 95/5).

2-Chloro-3-(5-bromopyridin-3-yl)indolizine-1-carbonitrile is prepared as described in Example 1 but starting with 1.46 g of 2-chloro-(3-pyridin-3-yl)-5,6,7,8-tetrahydroindolizine-1-carbonitrile in solution in 65 cm³ of xylene, 3.36 g of selenium oxide are added. 1.15 g of 2-chloro-3-(5-bromopyridin-3-yl)indolizine-1-carbonitrile are thus obtained in the form of a brown solid melting at 196° C.

2-Chloro-3-(5-bromopyridin-3-yl)-5,6,7,8-tetrahydroindolizine-1-carbonitrile is prepared according to the following method:

1.7 g of N-chlorosuccinimide are added to 3.32 g of 3-(5-bromopyridin-3-yl)-5,6,7,8-tetrahydroindolizine-1-carbonitrile in solution in 170 cm³ of acetonitrile. The mixture is heated at the reflux temperature of acetonitrile for 5 hours. The reaction mixture is evaporated to dryness under reduced pressure (2.7 kPa) at a temperature close to 60° C. The yellow oil obtained is taken up in 50 cm³ of water and the pH is brought to 8-9 by addition of powdered sodium hydrogen carbonate. The mixture is filtered and washed with twice 10 cm³ of water. 3.6 g of a yellow solid are obtained, which solid is chromatographed on a column 3.5 cm in diameter containing 100 g of silica (0.02-0.045). The elution is carried out with a mixture of dichloromethane/ethyl acetate (95/5), at a pressure of 150 kPa, collecting 20 cm³ fractions. The homogeneous fractions are combined and evaporated to dryness under reduced pressure (2.7 kPa) at a temperature close to 40° C. 1.8 g of a white solid are obtained, which solid is washed with 5 cm³ of acetonitrile. 1.46 g of 2-chloro-3-(5-bromopyridin-3-yl)-5,6,7,8-tetrahydroindolizine-1-carbonitrile are thus obtained in the form of a white solid melting at 162° C.

3-(5-Bromopyridin-3-yl)-5,6,7,8-tetrahydroindolizine-1-carbonitrile is prepared as described in Example 5 but starting with 15.89 g of sodium N-(5-bromonicotinoyl)piperidine-2-carboxylate, 9.93 g of para-toluene and 4.56 g of chloroacrylonitrile. 9.51 g of 3-(5-bromopyridin-3-yl)-5,6,7,8-tetrahydroindolizine-1-carbonitrile are thus obtained in the form of a beige solid melting at 148° C.

The sodium salt of N-(5-bromonicotinoyl)piperidine-2-carboxylic acid is prepared as described in Example 5 but starting with 23.15 g of ethyl N-(5-bromonicotinoyl)piperidine-2-carboxylate and 20.3 cm³ of 10 N sodium hydroxide. 15.89 g of sodium salt of N-(5-bromonicotinoyl)piperidine-2-carboxylic acid are thus obtained in the form of a white solid melting at 190° C.

Ethyl N-(5-bromonicotinoyl)piperidine-2-carboxylate is prepared according to the following method:

24.9 g of 1-(3-dimethylaminopropyl)-3-ethylcarbodiimide hydrochloride and 7.7 g of 1-hydroxybenzotriazole hydrate are added to a suspension of 17.3 g of ethyl 2-piperidinecarboxylate in 700 cm³ of dichloromethane and then 20.2 g of 5-bromonicotinic acid are added. 21.2 cm³ of triethylamine are added. The mixture is kept stirred at room temperature overnight. The reaction mixture is washed with 3 times 200 cm³ of water. The organic phase is then dried over magnesium sulphate and concentrated under reduced pressure (2.7 kPa). The oil obtained is filtered on a column 7 cm in diameter containing 343 g of silica (0.04-0.02). The elution is carried out with ethyl acetate, collecting 120 cm³ fractions. The homogeneous fractions are pooled and concentrated under reduced pressure (2.7 kPa). 823.15 g of ethyl N-(2-bromonicotinoyl)piperidine-2-carboxylate are thus obtained in the form of a yellow oil (Rf=0.61; thin-layer chromatography on silica gel; eluent ethyl acetate).

### EXAMPLE 8

2-Methyl-3-(pyridin-3-yl)indolizine-1-carboxamide is prepared according to the same method as that described in Example 2, but starting with 2.7 g of 2-methyl-(3-pyridin-3-yl)indolizine-1-carbonitrile and 2.29 g of potassium hydroxide in 200 cm³ of tert-butanol. 1.4 g of 2-methyl-3-(pyridin-3-yl)indolizine-1-carboxamide are thus obtained in the form of a cream-coloured solid melting at 180° C.

2-Methyl-(3-pyridin-3-yl)indolizine-1-carbonitrile is prepared according to the same method as that described in Example 3, but starting with 7.9 g of 2-methyl-(3-pyridin-3-yl)-5,6,7,8-tetrahydroindolizine-1-carbonitrile in solution in 350 cm³ of xylene and 25.86 g of selenium oxide. 2.9 g of 2-methyl-(3-pyridin-3-yl)indolizine-1-carbonitrile are thus obtained in the form of an orange-coloured solid melting at 140° C.

2-Methyl-(3-pyridin-3-yl)-5,6,7,8-tetrahydroindolizine-1-carbonitrile is prepared according to the following method:

16.4 cm³ of triethylamine are added to a cream-coloured suspension of 23.4 g of sodium N-nicotinoylpiperidine-2-carboxylate in 100 cm³ of 1,2-dichloroethane; a white suspension is obtained which is kept stirred at room temperature for 2 hours.

9.6 cm³ of 2-chloromethylacrylonitrile are added to a solution of 20 g of para-toluenesulphonyl chloride in 120 cm³ of 1,2-dichloroethane. An orange-coloured solution is obtained. This solution is added dropwise to the cream-coloured suspension obtained above. Next, 16.4 cm³ of triethylamine are added and the mixture is heated at the reflux temperature for 4 hours. The reaction mixture is kept stirred and at room temperature for 12 hours and then the mixture is washed with 3 times 500 cm³ of water. The aqueous phase is extracted with 500 cm³ of 1,2-dichloroethane and then the organic phase is dried over magnesium sulphate. The mixture is evaporated to dryness under reduced pressure (2.7 kPa) at a temperature close to 60° C. 21 g of a black oil are obtained, which oil is chromatographed on a column 9 cm in diameter containing 550 g of silica (0.02-0.045). The elution is carried out with a mixture of cyclohexane/ethyl acetate (50/50), at a pressure of 150 kPa, collecting 100 cm³ fractions. The homogeneous fractions are pooled and evaporated to dryness under reduced pressure (2.7 kPa) at a temperature close to 40° C. 10.41 g of 2-methyl-(3-pyridin-3-yl)-5,6,7,8-tetrahydroindolizine-1-carbonitrile are thus obtained in the form of an orange-coloured solid melting at 117° C.

The sodium salt of N-nicotinoylpiperidine-2-carboxylic acid is prepared as described in patent application EP 124,384.

2-Chlorocrotonitrile is prepared according to J. C POMMELET, C. NYNS, F. F LAHOUSSE, R. MERENYL and H. G VIEHE, Angew. Chem. Int. Ed. 21, 585 (1981).

### EXAMPLE 9

2-Cyano-3-(pyridin-3-yl)indolizine-1-carboxamide is prepared according to the method described in Example 2, but starting with 1.1 g of 2-cyano-3-pyridin-3-ylindolizine-1-carbonitrile and 0.89 g of potassium hydroxide in 100 cm³ of tert-butanol. 0.042 g of 2-cyano-3-(pyridin-3-yl)indolizine-1-carboxamide is thus obtained in the form of a beige solid melting at 258° C.

2-Cyano-3-(pyridin-3-yl)indolizine-1-carbonitrile is prepared as described in Example 1 but starting with 3.5 g of 2-cyano-3-(pyridin-3-yl)-5,6,7,8-tetrahydroindolizine-1-carbonitrile in the form of a cream-coloured solid melting at 258° C.

2-Cyano-3-(pyridin-3-yl)-5,6,7,8-tetrahydroindolizine-1-carbonitrile is prepared as described in patent application WO 98/25612.

The present invention also relates to the pharmaceutical compositions intended for the treatment and/or prophylaxis of conditions in which one or more viruses of the herpes family are involved and/or in which the cytokines, including TNF_{α}, are involved, containing a pyrrole derivative of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, optionally in the form of a salt, in the pure state or in the form of a combination with one or more compatible and pharmaceutically acceptable diluents or adjuvants.

The pharmaceutical compositions according to the invention are capable of slowing the progression to the disease or of reducing its severity in the infected subjects.

They are capable of preventing or slowing, in immunosuppressed subjects, the progression of subjects infected with a virus of the herpes family to a worsened state of the disease.

The pharmaceutical compositions according to the invention are also capable of inhibiting the replication of retroviruses and therefore of slowing the progression to the disease and of reducing its severity in the infected subjects. In particular, in the case of HIV infections, by inhibiting the replication of this virus, they are capable of slowing the progression to AIDS or of reducing its severity in the infected subjects. The pharmaceutical compositions according to the invention can be used for preventive or curative purposes. “Preventive” is understood to mean the preventing of progression in subjects exhibiting immunodeficiency and/or infected with retroviruses.

Of course, in the case of treatment in immunosuppressed individuals, the constitution of these compositions will be adapted to the specific case of the digestive tract of these subjects.

The compositions can be used by the oral, parenteral, topical or rectal route.

Sterile compositions for parenteral administration may be preferably solutions which are aqueous or nonaqueous, suspensions or emulsions. As solvent or vehicle, there may be used water, propylene glycol, polyethylene glycol, vegetable oils, in particular olive oil, injectable organic esters, for example ethyl oleate or other suitable organic solvents. These compositions may also contain adjuvants, in particular wetting, isotonizing, emulsifying, dispersing and stabilizing agents. Sterilization can be achieved in several ways, for example by aseptisizing filtration, by incorporating sterilizing agents into the composition, by irradiation or by heating. They can also be prepared in the form of solid sterile compositions which can be dissolved at the time of use in a sterile injectable medium.

As solid compositions for oral administration, there may be used tablets, pills, powders or granules. In these compositions, the active product according to the invention (optionally combined with another pharmaceutically compatible product) is mixed with one or more inert diluents or adjuvants, such as sucrose, lactose or starch. These compositions may also comprise substances other than diluents, for example a lubricant such as magnesium stearate.

As liquid compositions for oral administration, there may be used emulsions which are pharmaceutically acceptable, solutions, suspensions, syrups, elixirs containing inert diluents such as water or paraffin oil. These compositions may also comprise substances other than the diluents, for example wetting, sweetening or flavouring products.

The compositions for topical administration may be for example creams, ointments or lotions.

The compositions for rectal administration are suppositories or rectal capsules, which contain, in addition to the active ingredient, excipients such as cocoa butter, semisynthetic glycerides or polyethylene glycols.

In general, the doctor will determine the dosage judged most appropriate according to the age, weight and factors specific to the product and to the subject to be treated. Generally, in adults, the doses are between 25 and 2000 mg per day.

It has, furthermore, been shown that the pyrrole derivatives of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, act in synergy when they are combined with other antiviral agents active on viruses of the herpes family or when they are combined with certain anti-HIV agents. The present invention also relates to the combinations consisting of a pyrrole derivative of general formula (I) as defined above or in which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, and of an active ingredient known for its activity on viruses of the herpes family or else known for its anti-retrovirus activity, optionally in the presence of pharmaceutically acceptable excipients.

The agents known for their activity on viruses of the herpes family which may be combined are chosen from agents which are compatible and chemically inert towards the pyrrole derivative according to the invention. In a non-limiting manner, these agents are chosen, for example, from cidofovir, ganciclovir, foscarnet, GS930 and 1263W94 and the like.

The anti-HIV agents which can be combined are chosen from agents which are compatible and chemically inert towards the derivatives according to the invention. Without implying any limitation, these agents are chosen from inhibitors of reverse transcriptase [zidovudine (AZT), didanosine (DDI), dideoxycytidine (DDC), lamivudine (3TC), TIBO, neviparine, PMEA and the like], among the protease inhibitors [for example saquinovir, ABT-538, MK-639 and the like], or from tat and rev protein inhibitors.

The pharmaceutical compositions comprising such combinations are also within the scope of the present invention.

The following example given with no limitation being implied illustrates a composition according to the invention.

### EXAMPLE

| | |
|---|---|
| 2-Chloro-3-(pyridin-3-yl)indolizine-1-carboxamide | 25 mg |
| Magnesium stearate: 1% | 2 mg |
| ACDISOL: 1% | 2 mg |
| Colloidal silica: 0.5% | 1 mg |
| Lactose | 170 mg |

1. Pyrrole derivatives characterized in that they correspond to the general formula: 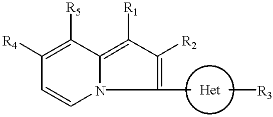
for which
R₁ is a carboxamide, cyano, carboxyl, alkyloxycarbonyl or acyl radical,
R₂ is a hydrogen or halogen atom, or a cyano, alkyl, alkyloxy, alkenyl or trihalomethyl radical,
R₃ is a hydrogen or halogen atom, or an alkyl or hydroxyl radical,
Het is a pyridyl, pyridyl N-oxide or thiazolyl radical,
R₄ is a hydrogen or halogen atom, or an alkylthio or alkyloxy radical, and
R₅ is a hydrogen atom, or a hydroxyl or alkyloxy radical,
it being understood that when R₃, R₄ and R₅ are hydrogen atoms and Het is a 2-pyridyl radical, then R₁ cannot be acetyl or methyloxycarbonyl and R₂ a hydrogen atom, or alternatively R₁ cannot be propionyl and R₂ methyl, the alkyl radicals being straight or branched and containing 1 to 4 carbon atoms and the alkenyl radicals being straight or branched and containing 2 to 4 carbon atoms, where appropriate in the form of their stereoisomers or mixtures thereof, as well as their salts when these exist.

2. Pyrrole derivatives according to claim 1, characterized in that they are chosen from the following list:
2-chloro-3-(pyridin-3-yl)indolizine-1-carbonitrile,
2-chloro-3-(pyridin-3-yl)indolizine-1-carboxamide,
3-(pyridin-3-yl)indolizine-1-carboxamide,
2,7-chloro-3-(pyridin-3-yl)indolizine-1-carboxamide,
methyl 2-methyl-3-(pyridin-3-yl)indolizine-1-carboxylate,
2-chloro-8-hydroxy-3-(pyridin-3-yl)indolizine-1-carboxamide,
2-chloro-3-(5-bromopyridin-3-yl)indolizine-1-carboxamide,
2-methyl-3-(pyridin-3-yl)indolizine-1-carboxamide,
2-cyano-3-(pyridin-3-yl)indolizine-1-carboxamide.

3. Process for the preparation of pyrrole derivatives according to claim 1, characterized in that there is prepared a nitrile intermediate of general formula: 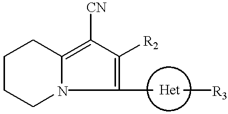
in which Het and R₃ are defined as above, and R₂ is a hydrogen atom or an alkyl or alkyloxy radical, by the action of an acrylic derivative of general formula: 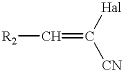
in which R₂ is defined as above, and Hal is a halogen atom on an acid of general formula: 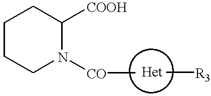
in which Het and R₃ are defined as above, followed by the steps of introducing, where appropriate, the radical R₂, aromatization, and introducing the radicals R₄ and/or R₅, and/or where appropriate converting the nitrile to an amide, an acid, an ester or an acyl radical, or alternatively, where appropriate, converting the ester radical to an acid or to an acyl radical, by any known methods which do not alter the rest of the molecule, and then where appropriate the product obtained is optionally separated into its stereoisomeric forms and/or the product obtained is converted to a salt.

4. Medicament, characterized in that it comprises at least one pyrrole derivative of general formula: 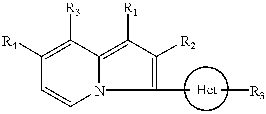
for which:
R₁ is a carboxamide, cyano, carboxyl, alkyloxycarbonyl or acyl radical,
R₂ is a hydrogen or halogen atom, or a cyano, alkyl, alkyloxy, alkenyl or trihalomethyl radical,
R₃ is a hydrogen or halogen atom, or an alkyl or hydroxyl radical,
Het is a pyridyl, pyridyl N-oxide or thiazolyl radical,
R₄ is a hydrogen or halogen atom, or an alkylthio or alkyloxy radical, and
R₅ is a hydrogen atom, or a hydroxyl or alkyloxy radical.

5. Pharmaceutical composition, characterized in that it comprises at least one pyrrole derivative as defined in claim 1 or alternatively for which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, in the pure state, optionally in combination with one or more antiviral agents active on viruses of the herpes family or alternatively in combination with one or more agents known for their antiretrovirus activity, and/or optionally in combination with one or more compatible and pharmaceutically acceptable diluents and/or adjuvants.

6. Synergizing combinations, characterized in that they comprise at least one pyrrole derivative as defined in claim 1 or for which when R₃, R₄ and R₅ are hydrogen atoms and Het is a 2-pyridyl radical, then R₁ is acetyl or methyloxycarbonyl and R₂ a hydrogen atom, or alternatively R₁ is propionyl and R₂ methyl, and at least one other antiviral agent active on viruses of the herpes family or alternatively at least one other antiretrovirus agent.
